(19) United States
(12) Patent Application Publication
(10) Pub. No.: US 2013/0196009 A1
(43) Pub. Date: Aug. 1, 2013

(54) ANTI-INFLAMMATORY AND ANTIOXIDANT COMPOSITION AND RELATED METHOD OF USE

(71) Applicant: Access Business Group International LLC, Ada, MI (US)

(72) Inventors: Arun Rajgopal, Grand Rapids, MI (US); Jatinder Rana, Grand Rapids, MI (US)

(73) Assignee: ACCESS BUSINESS GROUP INTERNATIONAL LLC, Ada, MI (US)

(21) Appl. No.: 13/751,433

(22) Filed: Jan. 28, 2013

Related U.S. Application Data
(60) Provisional application No. 61/592,355, filed on Jan. 30, 2012.

(51) Int. Cl. A61K 36/48; A61K 45/06

(52) U.S. Cl. CPC A61K 36/48; A61K 45/06
USPC 424/757

(57) ABSTRACT

A composition having at least one of a high antioxidant capacity, an anti-inflammatory effect and/or a DNA protection function is provided. The composition includes an extract from plants of the genus Caesalpinia, for example, Caesalpinia spinosa, also known as Tara. The Tara extract can be included in a nutritional supplement or other delivery vehicle and administered to a subject. Methods for using the composition also are provided.

### BACKGROUND OF THE INVENTION

The present invention relates to compositions that can provide an anti-inflammatory effect, an antioxidant function and/or aid in DNA protection.

Inflammation is usually directly related to traumatic injury, disease and/or conditions associated with aging. Inflammation sometimes is also associated with other conditions, such as gastrointestinal diseases, Alzheimer's disease, immune system deficiencies, some forms of cancer, as well as cardiovascular disease. Inflammation can be caused from a variety of conditions, environmental factors and/or genetics. Some examples of certain conditions include stress, environmental factors, obesity, diabetes, chronic infections and the like. Environmental factors that are usually linked to inflammation include diet or poor nutrition. Further, certain studies indicate that diets high in simple sugars and starches, as well as diets high in saturated trans fat, can be linked to or can cause certain types of inflammation.

Inflammation can also be linked to chemical oxidation that occurs in vivo. For example, oxidation of polyunsaturated fats can cause the formation of reactive oxygen species and the production of nitric oxide. These products can be associated with early phase inflammation and, if left unchecked, can eventually lead to chronic inflammation. Moreover, such reactive oxygen species can result in DNA damage and undesirable mutation.

A variety of commercially available nutritional supplements attempt to provide anti-inflammatory and antioxidant functionality. Many such supplements utilize compounds such as vitamin C, polyphenols, tocopherols and other natural compositions, usually derived from plant material. Although there are a variety of such nutritional supplements on the market, there still remains room for improvement, particularly with regard to the incorporation of natural sources into compositions that can provide antioxidant or anti-inflammatory effect.

### SUMMARY OF THE INVENTION

A composition provides at least one of an anti-inflammatory effect, an antioxidant function, a DNA protective function or a combination of the foregoing. The composition can include an effective amount of an extract derived from the plant genus, Caesalpinia, for example, Caesalpinia spinosa, which is also known as a “Tara”.

In one embodiment, a composition including Tara, for example, a Tara extract, can be used to counter inflammation in vivo. Optionally, the Tara can provide an increased antioxidant capacity, which in turn can neutralize or reduce free radicals, such as reactive oxygen species, that may be present in a subject. The composition further optionally can be used to protect DNA in vivo from, for example, oxidative damage caused by certain reactive oxygen species in the body of the subject.

In another embodiment, a method is provided for reducing or eliminating inflammation by administering the composition including Tara to a subject in an amount sufficient to inhibit or eliminate oxidation within various systems and cells of the subject. Optionally, the composition neutralizes and/or reduces free radicals, such as reactive oxygen species within various systems and cells of the subject.

In still another embodiment, a method is provided for treating inflammation by administering the composition to a subject in an amount sufficient to reduce or eliminate inflammation in the subject.

In yet another embodiment, a method is provided for protecting DNA, that is, preventing mutation and/or damage to DNA via certain reactive oxygent species, by administering the composition to a subject in an amount sufficient to protect DNA.

These and other objects, advantages and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a graph illustrating a dose-dependent reduction in DNA damage via administration of a current embodiment of the composition including Tara.

FIG. 2 is a graph illustrating a maximum effective concentration of Tara for DNA protection.

FIG. 3 is a graph illustrating inhibition of enzymes, known to be linked with inflammation, via administration of the current embodiment of the composition including Tara.

FIG. 4 is a graph illustrating the antioxidant capacity of the current embodiment of the composition including Tara relative to multiple major free radicals.

### DESCRIPTION OF THE CURRENT EMBODIMENTS

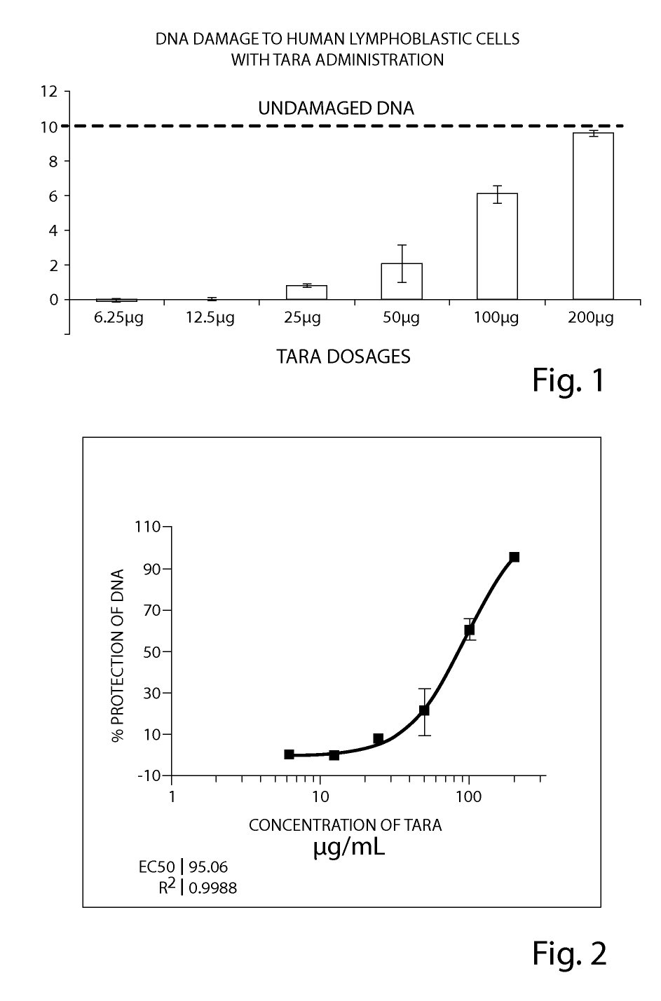

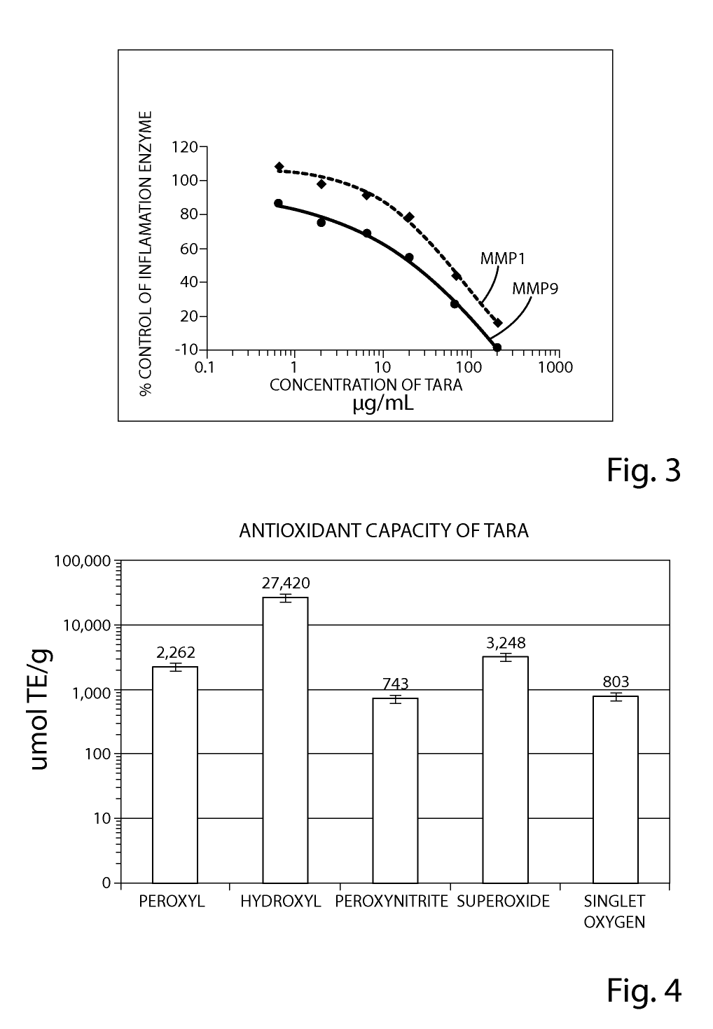

The current embodiments are based on the surprising discovery that extracts of genus Caesalpinia exhibit can increase DNA protection functionality, anti-inflammatory effects and antioxidant effect. The particular composition investigated included an extract of a plant from the genus, Caesalpinia, specifically Caesalpinia spinosa, which also is referred to as “Tara”, or alternatively as Molina, Kuntze or Spiny Holdback. In particular, as shown in FIG. 1, when administered to human lymphoblastic cells, the composition of Tara extract provided a dose-dependent positive effect on DNA damage protection. Likewise, as shown in FIG. 3, the composition of Tara extract in varying doses exhibited a dose-dependent positive effect on inhibiting inflammatory enzymes. As further illustrated in FIG. 4, the Tara extract composition illustrated a very high activity against multiple oxidative free radicals. Building on the discoveries herein, a current embodiment is directed to a composition that includes an extract of a plant of the genus Caesalpinia, for example, Caesalpinia spinosa, “Tara” in amount sufficient to provide at least one of an anti-inflammatory effect, an antioxidant function and/or aid in DNA protection in a subject, and optionally in a subject in need thereof.

As noted above, the composition can include extracts from Caesalpinia spinosa plants, however, it is contemplated that other plants of the Caesalpinia genus, such as Caesalpinia cacalaco, Caesalpinia californica, Caesalpinia gilliesii, Caesalpinia mexicana, Caesalpinia platyloba, and Caesalpinia pulcherrima also can provide at least one of an anti-inflammatory effect, an antioxidant function and/or aid in DNA protection in a subject.

Generally, the composition including the Tara extract can have a pH optionally between 6.0 to about 8.0, further optionally between about 6.5 to about 7.5, and further optionally a pH of about 7.0, or generally substantially neutral. The Tara extract used herein may be commercially obtained from various resources, including but not limited to, Yunnan Yimen Mingxin Tara Biotechnology Development Co., Ltd. of Yunan, China. In addition, any suitable Tara extracts can be obtained by using any conventional extraction techniques.

The Tara extract in the composition can be present in an amount optionally from about 0.001% to about 50% by weight of the composition, further optionally, in an amount of about 0.1% to about 30% by weight of the composition, and further optionally about 1% to about 10% by weight of the composition. Depending on the application, other amounts of Tara extract may be used.

The Tara extract, and/or other extracts from suitable plants of the Caesalpinia genus, can be used alone or in combination with one another, or with other ingredients, extracts, and/or excipients. Other suitable extracts can include extracts or ingredients chosen from rosehips, blueberry, blackberry, elderberry, cranberry, rosemary, clove, feverfew, nettle root, artichoke, olive extract, green tea extract (epigallocatechin gallate), grape seed extract, resveratrol, Aframomum melegueta, boswellia serrata extract, boswellia forte, ipriflavone, tocotrienols, evening primrose oil, INM-176, borage oil, hill oil, at least one type of xanthophyll (e.g., astaxanthin), green coffee extract (chlorogenic acid), and ferulic acid. Further, the extracts can be obtained from plant materials via a variety of methods including but not limited to those extraction methods disclosed in U.S. Pat. No. 7,897,184 to Rana et al, which is hereby incorporated by reference in its entirety.

The composition including the extract can further include one or more pharmaceutically acceptable excipients, for example, silicified microcrystalline cellulose, silicon dioxide, stearic acid, hydroxyl propyl methyl cellulose (HPMC), lactose, glucose, sucrose, corn starch, potato starch, cellulose acetate, ethyl cellulose and the like. The composition also can include diluents and other additives, such as one or more pharmaceutically acceptable binding agents, fillers, support agents, thickening agents, taste-improving agents, coloring agents, preservatives, proteins, sweeteners, anti-microbials, chelating agents, inert gases, stabilizers, regulators, emulsifiers or mixtures thereof, depending on the form of the composition employed.

The composition can be in a variety of forms having different delivery systems. For example, the composition can be processed and included in tablets, capsules, gel tabs, lozenges, strips, granules, powders, concentrates, solutions, lotions, cremes, suspensions and/or combinations thereof. The composition can be administered to a subject orally, by ingestion, or sublingually in the oral cavity, or it can be administered via the respiratory tract, for example, via a spray, mist or aerosol.

The composition can be formulated individually or in combination with other foods or nutritional supplemental foods and the like. As one example, the composition can include one portion of the composition in tablet form and another portion of the composition administered in capsule form.

Further, the composition can be administered in varying dosages in various increments of time determined by the condition or the desired effect of the composition. For example, the composition can be administered in multiple successive dosages, spaced as frequently as about 6 to about 12 hours apart, or as long as about 1, 2 or 3 weeks apart. Daily dosages may be administered for 1, 2 or 3 times a day for a period to obtain systematic relief from certain conditions such as inflammation. The dosages may be adjusted to maintain the relief from such systems. Administration of the composition can be for a period of weeks or months, or optionally for the long term, as part of a nutritional supplement regimen.

Generally, the composition can be administered in any suitable regimen so that the desired outcome is achieved, for example, inflammation subsides, reactive oxygen species and/or free radicals are reduced, and/or mutation or damage to DNA of the subject is reduced. Again, the frequency of administration of the composition can depend on several factors including the desired level of inflammation reduction or elimination, antioxidant function and/or DNA protection.

In general, the composition can be administered to a subject suffering from inflammation, either acute or chronic. The subject can be identified as having the inflammation via a variety of conventional tests or diagnostic procedures for inflammation. For example, the composition can be used to reduce the inflammatory response associated with diseases and/or abnormal conditions. Optionally, the composition might be used to reduce the symptoms associated with and/or treat arthritis, cardiovascular conditions, osteoporosis, Alzheimer's disease, and an inflammatory response to a traumatic injury or other abnormal conditions or diseases associated with inflammation, such as pain and stiffness. Further optionally, the composition might be administered in a therapeutically effective amount to a subject having at least one of arthritis, cardiovascular conditions, osteoporosis, an inflammatory response, and/or conditions or diseases associated with inflammation, such as pain and/or stiffness.

### EXAMPLE 1

To analyze the effect of the composition, and in particular the Tara extract, on DNA protection an assay was performed. To test DNA protection, a single cell gel electrophoresis assay, also known as comet assay, was performed to detect DNA damage of eukaryotic cells, and in particular human lymphoblastic cells, pre-treated with the Tara extract. The comet assay is described in Singh et al (1988), Experimental Cell Research, 175(1):184-191, which is hereby incorporated by reference. In the assay, human lymphoblastic cells were prepared according to the standardized method for a comet assay as presented in Singh et al., and pretreated with different amounts of Tara extract, specifically, 6.25 μg, 12.5 μg, 25 μg, 50 μg, 100 μg, and 200 μg of Tara extract for one hour. The cells were washed and treated 100 μM of hydrogen peroxide for 20 minutes. The cells treated with the hydrogen peroxide exhibited different levels of DNA damage depending on the pre-treatment with the Tara extract.

As can be seen in FIG. 1, the Tara extract exhibited a concentration-dependent DNA protection function. For example, at lower levels of concentration, the Tara extract did not protect the cells to a significant extent, and accordingly, the hydrogen peroxide damaged the DNA somewhat. However, the cells treated with the higher doses of Tara extract were protected so that the DNA of those cells remained generally undamaged as shown to the right of FIG. 1. Generally, dosages of Tara extract administered to the cells at levels optionally greater than about 90 μg to about 100 μg, further optionally greater than 200 μg, exhibited very good DNA protection.

Based on this finding, it is believed that a composition as described herein can be used to provide DNA damage protection within a subject to whom the composition is administered, or generally can protect DNA from abnormally mutating or becoming damaged so that it cannot replicate appropriately in a subject.

FIG. 2 illustrates the effective concentration EC50 value of the Tara extract in the comet assay discussed above and shown in FIG. 1. There, it can be seen that the EC50 value of the Tara extract is 95.06 μg/mL which concentration provides DNA protection under the comet assay. It is believed that concentrations of administered Tara extract in the optional range of about 80 μg/mL to about 120 μg/mL, further optionally greater than 120 μg/mL, and even further optionally greater than 200 μg/mL, can provide DNA protection under the comet assay.

### EXAMPLE 2

To analyze the effect of the composition, and in particular the Tara extract, on inflammation, the following assay was performed. Generally, the inhibition of certain enzymes responsible for inducing inflammation was measured to determine the effectiveness of Tara extract. In particular, MMP1 and MMP9 enzyme inhibition was measured in the assay. Conventionally purified recombinant MMP protein, commercially available from EMD Biosciences of North America, was combined with fluorogenic peptide substrate IX, commercially available from R&D Systems of Minneapolis, Minn., in the presence of varying concentrations of diluted Tara extracts, as indicated by the points on the graph at FIG. 3. The conversion of the fluorogenic peptide substrate over time was monitored with a fluorescent plate reader, particularly a SpectraMax M5 plate reader, which is commercially available from Molecular Devices, LLC of Sunnyvale, Calif., United States. The data were collected, and the Vmax for each well and the percent control (that is, the percent inhibition of the MMPs) were calculated and compared to the enzyme, plus the substrate maximum response control.

Based on the results illustrated in FIG. 3, the Tara extract effectively inhibited, for example, measured inflammation-associated enzymes, MMP1 and MMP9, particularly with increasing concentrations of the Tara extract. Therefore, it is believed that the composition with Tara extract likely can effectively reduce and/or inhibit an inflammatory response in a subject.

### EXAMPLE 3

To analyze the antioxidant effect of the composition, and in particular the Tara extract, its ability to neutralize multiple major free radicals was investigated. In particular, Tara extract in varying concentrations was tested in conventional peroxyl, hydroxyl, peroxynitrite, superoxide and single oxygen assays. The assays included individual testing of peroxyl radical, hydroxyl radical, superoxide radical anion, singlet oxygen, and peroxynitrite anion. The assays were performed at Brunswick Laboratories, of Southborough, Mass. The antioxidant capacity for each parameter was quantified using area-under-the-curve kinetic analysis, such as those disclosed in Dubost, Ou, & Beelman, 2007; Huang, Ou, Hampsch-Woodill, Flanagan, & Prior, 2002; Ou, et al., 2002; Ou, Hampsch-Woodill, & Prior, 2001; Zhang, et al., 2009, which are hereby incorporated by reference. Each assay involved the use of a reagent to generate the specific RNOS species, a target molecule for oxidation that also served as an analytical probe for measurement, and the sample antioxidant to be assessed. Each assay was standardized to Trolox, and reported in units of micromole Trolox equivalents per gram of sample (μmol TE/gram). Huang, D., Ou, B., Hampsch-Woodill, M., Flanagan, J. A., & Prior, R. L. (2002), High-throughput assay of oxygen radical absorbance capacity (ORAC) using a multichannel liquid handling system coupled with a microplate fluorescence reader in 96-well format. J Agric Food Chem, 50, 4437-4444, which is incorporated by reference herein.

As shown in FIG. 4, the composition including the Tara extract exhibited a high anti-oxidant capacity against the reactive oxygen species peroxyl, hydroxyl, peroxynitrite, superoxide and single oxygen. As an illustration, the Tara extract antioxidant capacity is more than 15 times that of cranberry, and more than 3 times that of elderberry, when analyzed under similar panels of assays. Accordingly, it is believed that when administered to a subject, the composition including the Tara extract will likewise exhibit a significant anti-oxidant effect.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z ; and Y, Z.

The embodiments of the invention in which an exclusive property or privilege is claimed are defined as follows:

1. A composition for administration to a subject, the composition comprising an extract from plants of the genus Caesalpinia in an amount effective to reduce inflammation in the subject.

2. The composition of claim 1 wherein the extract is obtained from Caesalpinia spinosa.

3. The composition of claim 2 wherein the extract is present in an amount of about 0.001% to about 50% by weight of the composition.

4. The composition of claim 3 wherein the extract is present in an amount of about 1% to about 10% by weight of the composition.

5. The composition of claim 4 comprising an excipient and at least one other extract, wherein the composition is in the form of a nutritional supplement administered to the subject on a periodic basis as part of a nutritional supplement regimen.

6. A composition for administration to a subject, the composition comprising an extract from plants of the genus Caesalpinia in an amount effective to provide an anti-oxidant effect in the subject.

7. The composition of claim 4 wherein the extract is obtained from Caesalpinia spinosa.

8. The composition of claim 5 wherein the extract is present in an amount of about 0.001% to about 50% by weight of the composition.

9. The composition of claim 8 wherein the extract is present in an amount of about 1% to about 10% by weight of the composition.

10. The composition of claim 9 comprising an excipient and at least one other extract wherein the composition is in the form of a nutritional supplement administered to the subject on a periodic basis as part of a nutritional supplement regime.

11. A composition for administration to a subject, the composition comprising an extract from plants of the genus Caesalpinia in an amount effective to at least one of inhibit and reduce damage to DNA of the subject.

12. The composition of claim 7 wherein the extract is obtained from Caesalpinia spinosa.

13. The composition of claim 8 wherein the extract is present in an amount of about 0.001% to about 50% by weight of the composition.

14. The composition of claim 8 wherein the extract is present in an amount of about 1% to about 10% by weight of the composition.

15. The composition of claim 9 comprising an excipient and at least one other extract wherein the composition is in the form of a nutritional supplement administered to the subject on a periodic basis as part of a nutritional supplement regime.

16. A method for reducing inflammation in a subject in need thereof comprising:
identifying a subject having inflammation;
administering a composition including an extract from plants of the genus Caesalpinia to the subject in an amount effective to reduce the inflammation of the subject.

17. A method for reducing oxygen free radicals in a subject comprising:
administering a composition including an extract from plants of the genus Caesalpinia in an amount effective to reduce the reactive oxygen species present in the subject.

18. A method for protecting DNA in a subject comprising:
administering a composition including an extract from plants of the genus Caesalpinia in an amount effective to the subject to reduce or impair at least one of DNA damage and DNA mutation.
